(19) United States
(12) Patent Application Publication
(10) Pub. No.: US 2020/0146554 A1
(43) Pub. Date: May 14, 2020

(54) SCANNING AND TRACKING MONITORING APPARATUS AND METHOD

(71) Applicant: Danmarks Tekniske Universitet, Kgs. Lyngby (DK)

(72) Inventors: Oline Vinter OLESEN, Herlev (DK); Claus BENJAMINSEN, Brønshøj (DK)

(21) Appl. No.: 16/061,432

(22) PCT Filed: Dec. 14, 2016
(86) PCT No.: PCT/EP2016/081032

§ 371 Date: Jun. 12, 2018

(30) Foreign Application Priority Data
Dec. 14, 2015 (EP) 15199948.9

(51) Int. Cl. A61B 5/00; A61B 5/055; A61B 6/03; A61B 6/00; G01R 33/28; G01R 33/565

(52) U.S. Cl. CPC A61B 5/0037; A61B 5/055; A61B 6/037; A61B 5/721; G01R 33/56509; A61B 5/742; A61B 6/5264; A61B 6/461; G01R 33/283; A61B 5/7405

(57) ABSTRACT

Disclosed is a scanning monitoring apparatus for medical imaging, the scanning monitoring apparatus comprising a controller unit and a display, wherein the controller unit during a scanning session is configured to obtain tracking data of a subject in a medical scanner, obtain scanner data indicative of operating parameters of the medical scanner; determine an output of a verification function based on the tracking data and the scanner data; and control the scanning monitoring apparatus according to the output of the verification function. A notification signal may be provided if the output is indicative of an erroneous scanning.

The present disclosure relates to a scanning monitoring apparatus and related method, in particular to a scanning monitoring apparatus for medical imaging, such as Magnetic Resonance Imaging (MRI) and/or Positron Emission Tomography (PET), and/or Computed Tomography (CT) and/or Magnetoencephalography (MEG), and radiotherapy.

### BACKGROUND

Motion tracking methods for motion correction in medical imaging have been investigated intensively during the past years but a functional clinical tool for verified motion detection during biomedical scan acquisition does not exist.

Motion during medical image acquisition degrades the quality of the medical images. Poor quality of medical images may result in costly rescans and worst case in wrong diagnosis and treatment. Optimally, the medical imaging data should be corrected for any motion; however known tracking methods suffer from reliability and therefore reduce the usability in clinical practice. Further, motion correction methods are in general specified to the image acquisition of the medical scanner and the object being scanned.

Medical scanner driven motion tracking methods developed for medical brain imaging generally suffer from an inability to obtain sufficiently high temporal and spatial resolution at the same time. Further, the high resolution of modern medical scanners—down to tenths of a millimeter for Magnetic Resonance Imaging (MRI) and a few millimeters for Positron Emission Tomography (PET)—sets strict requirements to motion tracking estimation. Most external tracking systems use markers attached to the subjects head. Marker-based systems introduce a critical source of errors where motion of the marker may not represent motion of the patient. This problem becomes significant due to the limited information available. There is no information to verify if the tracking correctly registered patient motion or only motion of the marker.

Markerless tracking methods based on 3D surface representation, e.g. using structured light, depth sensors, and stereo vision systems, are complex with high risk of unpredictable errors.

### SUMMARY

There is a need for apparatus and methods providing an improved clinical tool for motion management in medical imaging/biological imaging based on tracking from 3D surface information.

A scanning monitoring apparatus for medical imaging is disclosed, the scanning monitoring apparatus comprising a controller unit and a display, wherein the controller unit is, during and/or before a scanning session, configured to obtain tracking data of a subject in a medical scanner, wherein to obtain tracking data optionally comprises to determine one or more quality parameters indicative of tracking data quality and to include the one or more quality parameters in the tracking data; obtain scanner data indicative of one or more operating parameters of the medical scanner; determine an output of a verification function based on the tracking data and/or the scanner data; and control the scanning monitoring apparatus according to the output of the verification function.

Further, a method of operating a scanning monitoring apparatus for medical imaging is disclosed, the scanning monitoring apparatus comprising a controller unit and a display, wherein the method comprises, during a scanning session, obtaining tracking data of a subject in a medical scanner, wherein obtaining tracking data optionally comprises determining one or more quality parameters indicative of tracking data quality and including the one or more quality parameters in the tracking data; obtaining scanner data indicative of operating parameters of the medical scanner; determining an output of a verification function based on the tracking data and the scanner data; and controlling the scanning monitoring apparatus according to the output of the verification function.

It is an important advantage of the present invention that the number of scanning sessions can be reduced by improving the quality of the medical images. Errors or poor image quality can be spotted immediately during a scanning session which enables the operator to repeat a scanning in the same scanning session without image reconstruction and inspection. Thus manual image inspection and scan time can be reduced. Further a person being scanned may avoid being recalled for a new scanning session at a later point in time which is highly desired, both with respect to optimum usage of scanning resources and reduced risk of the person being scanned. It is a further advantage of the present invention that the motion tracking may be made clinical applicable for control of the scanning, for example by allowing for motion correction and quality control.

Further, the present disclosure contributes to improved patient security, since the risk that buggy or erroneous biological images form basis for diagnosis and treatment is heavily reduced. Further, better diagnosis and treatment may be obtained.

The present disclosure presents simultaneous or realtime tracking and monitoring combined from 3D surface scanning. Verified tracking data plus scanner info may enable output from a verification function guiding the image acquisition. Accordingly, the quality of scanning sessions may be improved by enabling an operator of a medical scanner to monitor the scanning environment in an easy and convenient way. The verified tracking information may also be given directly to the medical scanner. It is envisaged that data also may be stored to be used for post-scanning correction.

### BRIEF DESCRIPTION OF THE DRAWINGS

The above and other features and advantages will become readily apparent to those skilled in the art by the following detailed description of exemplary embodiments thereof with reference to the attached drawings, in which:

FIG. 1 schematically illustrates an exemplary scanning monitoring apparatus,

FIG. 2 schematically illustrates a display with exemplary first and second representations,

FIG. 3 schematically illustrates an exemplary method,

FIG. 4 schematically illustrates an example of obtaining tracking data,

FIG. 5 illustrates operation of an exemplary controller unit, and

FIG. 6 schematically illustrates a display with exemplary first and second representations.

### DETAILED DESCRIPTION

Various embodiments are described hereinafter with reference to the figures. Like reference numerals refer to like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

Throughout, the same reference numerals are used for identical or corresponding parts.

The scanning monitoring apparatus combines tracking data and scanner data, effectively improving the quality of a scanning of a subject during a scanning session. The scanning monitoring apparatus may thus be a scanning and tracking monitoring apparatus. A scanning session may be defined as the time period from when the scanning subject is put in the scanner to when the scanning subject is removed from the scanner. The subject is also denoted scanning subject.

The scanning monitoring apparatus comprises a controller unit and a display. The display comprises a display controller and display face. The display may be a computer screen, e.g. an LED display, such as an OLED display, or a Liquid Crystal Display (LCD).

The controller unit is configured to obtain tracking data of a subject in a medical scanner during a scanning session. The tracking data may comprise image control data of one or more image projectors, such as image settings. The tracking data may comprise 3D representation data of an imaging region of the subject, e.g. from any sensor system capable of providing, or configured to provide, 3D surface representation data of an imaging region of the subject, including a surface scanner, a stereo-vision or a multiple-vision camera system, a depth sensor, a time-of-flight camera and/or a structured light imaging system, etc. The tracking data may comprise image data from one or more cameras or detectors. The tracking data may comprise scanner driven tracking data e.g. from image navigators, k-space navigators, and centroids. The tracking data may comprise one or more quality parameters indicative of tracking quality. The quality parameter(s) may be based on 3D representation data, e.g. comprising a 3D point set. The tracking data may comprise displacement data.

To obtain tracking data may comprise to determine one or more quality parameters indicative of tracking data quality and to include one or more quality parameters in the tracking data.

To obtain tracking data may comprise to apply a weighting function and/or a filtering function on the raw tracking data, e.g. 3D surface representation data, based on raw tracking data and/or on scanner data and/or user input data. The raw tracking data, which the weighting function is based on, may comprise 2D images, 3D representation data, such as 3D point sets, including for example current representation data, previous representation data from which e.g. distances to neighbor points and point normals may be extracted. The scanner data and/or the user input data, which the weighting function is based on, may comprise regions of interests (e.g. to mask out MR coils), or a model of the scanning subject. The one or more quality parameters indicative of tracking data quality may be based on the output of the weighting/filtering function.

To obtain tracking data may comprise to determine displacement data indicative of subject motion and to include displacement data or at least parts thereof in the tracking data. Thus, the controller unit may be configured to determine displacement data, e.g. based on one or more reference points or groups of reference points. The reference points may be derived from the scanning subject data and/or be obtained via the user interface. The one or more quality parameters in the tracking data may be based on the displacement data.

The controller unit is configured to obtain scanner data indicative of operating parameters of the medical scanner during and/or before the scanning session. Scanner data may be obtained via a scanner interface connected to the medical scanner and/or a user interface. The user interface may allow an operator to feed scanner data into the scanning monitoring apparatus. Scanner data may be obtained from a scanning subject database comprising one or more scanning subject parameters. The scanner data may comprise a scanning type identifier indicative of the scanner type (PET, MR, CT, MEG or any combination) and/or scanner model. Different types of medical scanners may have different requirements to the subject that is scanned.

The controller unit may further be configured to obtain scanning subject parameters.

The controller unit may be configured to obtain user input data via a user interface. The user data may comprise a motion threshold, such that for example a notification is provided when the motion threshold is reached.

The scanner data may comprise one or more operating parameters, such as a first operating parameter and a second operating parameter, of the medical scanner.

The one or more operating parameters of the medical scanner may comprise a first operating parameter or a set of first operating parameters, wherein the first operating parameter(s) may be indicative of a scanning mode. A scanning mode may include specific settings and operating parameters for the scanner. As an example first operating parameters may for MRI include strength of magnet, bore size, contrast agents, etc. A scanning mode may for MRI be a conventional MRI, diffusion tensor imaging, functional MRI (fMRI), Blood-oxygen-level dependent, diffusion weighed imaging (DWI), MRI spectroscopy, MR angiography, etc. A scanning mode may for PET be imaging of a stationary tracer, dynamic tracer, different tracers e.g. fluorodeoxyglucose (FDG), and different isotopes e.g. fluorine (18F) and carbon (11C, 13C, 14C). A scanning mode may be indicative of an MRI scanning sequence, e.g. MPRAGE, EPI, FLAIR, STIR, T1-weighted, or T2-weighted.

The one or more operating parameters of the medical scanner may comprise a second operating parameter or a set of second operating parameters, wherein the second operating parameter(s) may be indicative of start of scan acquisition.

The one or more operating parameters of the medical scanner may comprise a third operating parameter or a set of third operating parameters, wherein the third operating parameter(s) may be indicative of end of scan acquisition.

The scanner data may comprise one or more scanning subject parameters of the subject that is scanned in the scanning session.

The one or more scanning subject parameters of the subject may comprise a first scanning subject parameter indicative of the age and/or weight of the scanning subject.

The one or more scanning subject parameters of the subject may comprise a second scanning subject parameter indicative of the examination to be performed on the scanning subject.

The one or more scanning subject parameters of the subject may comprise a third scanning subject parameter indicative of the disease of scanning subject, such as tumor/lesion, Alzheimer's disease, neurotic diseases, etc. . . . .

The one or more scanning subject parameters of the subject may comprise a fourth scanning subject parameter indicative of scanning subject symptoms, such as pain, dementia, or tremor.

The one or more scanning subject parameters of the subject may comprise a fifth scanning subject parameter indicative of scanning subject consciousness level, such as sedated, awake, sleeping, etc.

The one or more scanning subject parameters of the subject may comprise a sixth scanning subject parameter indicative of one or more of position, shape and size of a scanning volume to be examined. For example, where a tumor is within a part of the brain of a scanning subject, such as the left/right frontal lobe, the left/right parietal lobe, left/right temporal lobe, the speech center, the occipital lobe, the hearing center, cerebellum, brain stem, or cerebrum or parts thereof, the sixth scanning subject parameter may be indicative of a part of the brain to be examined.

The one or more scanning subject parameters of the subject may comprise a seventh scanning subject parameter indicative of injection of contrast agents and/or PET tracers.

The one or more scanning subject parameters of the subject may comprise an eighth scanning subject parameter indicative of injection dose and time.

The controller unit is configured to determine an output of a verification function based on the tracking data and the scanner data during and/or before the scanning session. The verification function may select and apply a verification function based on the scanner data, such as one or more operating parameters of the medical scanner and/or one or more scanning subject parameters. The verification function may select a verification function from a plurality of verification functions. The verification function may apply a first verification function if the first operating parameter of the medical scanner is indicative of a first scanning mode. The verification function may apply a second verification function if the first operating parameter of the medical scanner is indicative of a second scanning mode. A verification function may calculate a parameter based on the tracking data and/or the scanner data. A verification function may determine if input data, an input value or the calculated parameter fulfils a criterion, e.g. if the input value is larger than a threshold value, if the input value is less than a threshold value or if the input value is equal to a value.

The output of the verification function may be indicative of corrupted tracking data, e.g. if a quality parameter of the one or more quality parameters indicative of tracking data quality does not fulfil a tracking data quality criterion. The verification function may verify the tracking data, e.g. by applying one or more tracking data criteria to the tracking data. The output of the verification function may be indicative of high quality of tracking data, e.g. if a quality parameter of the one or more quality parameters indicative of tracking data quality fulfils a tracking data quality criterion.

The output of the verification function may be based on the tracking data, e.g. raw tracking data, for example data including 2D and 3D surface representation data, and/or displacement data. The output of a verification function may be based on the tracking data, e.g. one or more quality parameters indicative of tracking data quality. It is an advantage of the present invention that an operator of the medical scanner is able to monitor tracking data quality. In particular, the apparatus allows an operator to distinguish between tracking data indicating erroneous scanning due to subject motion or due to poor tracking quality or errors in the tracking data. Errors in the tracking data may for example include occlusion of the subject, presence of foreign object, unwanted deformation and/or interference from light.

The first verification function may comprise to determine if tracking data fulfils a first error criterion, e.g. if displacement is larger than a first threshold value, and wherein the output of the verification function is set to a value indicative of an erroneous scanning if the first error criterion is met.

The second verification function may comprise to determine if tracking data fulfils a second error criterion, e.g. if displacement is larger than a second threshold value, and wherein the output of the verification function is set to a value indicative of an erroneous scanning if the second error criterion is met.

The controller unit is configured to control the scanning monitoring apparatus according to the output of the verification function, e.g. during and/or before the scanning session.

To control the scanning monitoring apparatus according to the output of the verification function may comprise to provide a notification signal or a plurality of notification signals if the output is indicative of an erroneous scanning. This allows an operator of the medical scanner to optimize the scanning session immediately or at least during the scanning session, in turn reducing the number of additional scanning sessions. The controller unit may be configured to select the notification signal from a plurality of notification signals based on the output of the verification function. Different types of notification signals may be selected for different types of erroneous scanning.

The scanning monitoring apparatus may comprise a loudspeaker. To provide a notification signal optionally comprises to provide an audio signal with the loudspeaker. An audio signal may be advantageous in that the chance of an operator noting an audio signal may be higher than the chance of noting other types of notification signals.

The controller unit may be configured to select the audio signal from a plurality of audio signals based on the output of the verification function. Thus, the scanning monitoring apparatus is able to notify different types of erroneous scanning.

To provide a notification signal may comprise to provide a visual alarm signal on the display. e.g. by sending a control signal from the controller unit to the display.

To control or controlling the scanning monitoring apparatus according to the output of the verification function may comprise to determine a notification scheme including a plurality of notification signals based on the output of the verification function. To control or controlling the scanning monitoring apparatus according to the output of the verification function may comprise to provide the notification scheme, optionally if the notification scheme comprises at least one notification signal.

Examples of notification signals are:
audio signal presented to operator, e.g. via loudspeaker of scanning monitoring apparatus.
one or more error indicators in first representation and/or in second representation.
control signal to medical scanner indicative of stop scanning, e.g. in case it is detected that the scanning subject is crawling out of the scanner or similar serious event)
control signal to medical scanner indicative of restart of scanning, e.g. if it is known that the current scanning session is of crucial importance, and the motion is very severe, or simple if the scanning just started and restarting can be done with almost no loss of time.
control signal to medical scanner indicative of rescan of slice (redo part of the scanning)
mark scanning result as motion corrupted, e.g. if there is no time to redo the scanning, and no motion correction methods exist, then the scanning images can at least be marked as being motion corrupted.
mark scanning result for retrospective, or potential retrospective, motion correction
mark scanning result if not motion corrupted.

Thus, the notifications may be provided to an operator at the time of scanning, furthermore, selected notifications may be provided to the subject, e.g. to indicate that motion should be reduced. Notifications may also be stored with the resulting scanning results, so as to indicate at a later stage whether data should be retrospectively corrected, such as retrospectively motion corrected, whether data may be used without correction, such as without motion correction, etc.

The scanning monitoring apparatus may provide feedback to the medical scanner. Accordingly, to control the scanning monitoring apparatus according to the output of the verification function may comprise to determine a first control signal based on the output of the verification function and/or to transmit a first control signal to the medical scanner. The first control signal may be indicative of stop scanning if the output is indicative of a first error.

Further, to control the scanning monitoring apparatus according to the output of the verification function may comprise to determine a second control signal based on the output of the verification function and/or to transmit a second control signal to the medical scanner. The second control signal may be indicative of scanning restart if the output is indicative of a second error. The restart may include a restart of the entire scanning session, a restart of the last scan, a redo of the last performed action, etc.

The scanning monitoring apparatus may provide improved representation of the results of the scanning session to an operator during the scanning session. To control the scanning monitoring apparatus according to the output of the verification function may comprise to determine and/or provide a first representation based on the tracking data. The controller unit may be configured to determine and/or provide the first representation based on the scanner data, e.g. one or more scanning subject parameters, and/or the user input data. The controller unit may be configured to provide the first representation to the display. The controller unit may be configured to to store the first representation in a memory. The first representation may comprise a graphical representation of displacement (translation and/or rotation) of the subject, e.g. with respect to a reference point or group of reference points of the subject and/or as a function of time. The graphical representation of displacement may comprise a first graphical representation. The first graphical representation may be of total 3D displacement in distance units. The first graphical representation may indicate translational movement of the subject, a first point with a fixed relation to the subject or a first group of points with a fixed relation to the subject, e.g. with respect to a reference point or group of reference points of the subject and/or as a function of time. The first graphical representation may indicate rotational movement of the subject, a first point with a fixed relation to the subject or a group of first points with a fixed relation to the subject, e.g. with respect to a reference point or group of reference points optionally with a fixed relation to the subject and/or as a function of time.

The graphical representation of displacement may comprise a second graphical representation. The second graphical resentation may be of first displacement along a first axis as a function of time. The second graphical representation may indicate translational movement of the subject, a second point with a fixed relation to the subject or a group of second points with a fixed relation to the subject, e.g. with respect to a reference point or group of reference points of the subject and/or as a function of time. The second graphical representation may indicate rotational movement of the subject, the first point with a fixed relation to the subject, a second point with a fixed relation to the subject, the group of first points with a fixed relation to the subject, or a group of second points with a fixed relation to the subject, e.g. with respect to a reference point or group of reference points optionally with a fixed relation to the subject and/or as a function of time. The first graphical representation may indicate any combination of translational and rotational movement of the subject, a first point with a fixed relation to the subject or a first group of points with a fixed relation to the subject, e.g. with respect to a reference point or group of reference points optionally with a fixed relation to the subject and/or as a function of time.

The graphical representation of displacement may comprise a third graphical representation. The third graphical representation may be of second displacement along a second axis as a function of time. The third graphical representation may indicate translational movement of the subject, a third point with a fixed relation to the subject or a group of third points with a fixed relation to the subject. e.g. with respect to a reference point or group of reference points with a fixed relation to the subject and/or as a function of time. The third graphical representation may indicate rotational movement of the subject, a third point with a fixed relation to the subject or a group of third points with a fixed relation to the subject. e.g. with respect to a reference point or group of reference points optionally with a fixed relation to the subject and/or as a function of time.

The graphical representation of displacement may comprise a fourth graphical representation. The fourth graphical representation may be of third displacement along a third axis as a function of time. The fourth graphical representation may indicate translational movement of the subject, a fourth point with a fixed relation to the subject or a group of fourth points with a fixed relation to the subject, e.g. with respect to a reference point or group of reference points with a fixed relation to the subject and/or as a function of time. The fourth graphical representation may indicate rotational movement of the subject, a fourth point with a fixed relation to the subject or a group of fourth points with a fixed relation to the subject, e.g. with respect to a reference point or group of reference points optionally with a fixed relation to the subject and/or as a function of time.

The controller unit may be configured to determine one or more indicators based on the output of the verification function and/or the scanner data. The one or more indicators may be based on the tracking data. The controller unit may be configured to include the one or more indicators or at least a part thereof in the first representation. The one or more indicators may comprise a first indicator or a set of first indicators indicative of start of scan acquisition. The one or more indicators may comprise a second indicator or a set of second indicators indicative of end of scan acquisition. The one or more indicators may comprise an error indicator or a set of error indicators indicative of erroneous scanning. The one or more indicators may comprise a third indicator or a set of third indicators indicative of scanning mode. The one or more indicators may comprise a fourth indicator or a set of fourth indicators indicative of one or more thresholds applied in the verification function. The one or more indicators may comprise one or more stop indicators indicative of stop of scan acquisition. The one or more indicators may comprise a resume indicator indicative of scan acquisition being resumed. The one or more indicators may comprise a pause indicator indicative of scan acquisition being paused.

The one or more indicators may indicate at which time the corresponding event occurred. Different indicators may have the same or different size. Different indicators may have the same or different shape. Different indicators may have the same or different color. The first indicator(s) may have a first color and a first shape. The second indicator(s) may have a second color and a second shape. The second color may be different from the first color. The third indicator(s) may have a third color and a third shape.

To control the scanning monitoring apparatus according to the output of the verification function may comprise to determine and/or provide a second representation based on the tracking data. The controller unit may be configured to determine and/or provide the second representation based on the scanner data, e.g. one or more scanning subject parameters, and/or the user input data.

The controller unit may be configured to provide the second representation to the display and/or to store the second representation in a memory. The second representation may comprise a surface representation of a surface of the scanning subject. The second representation may comprise a reference surface representation of a surface of the scanning subject. The second representation may comprise one or more indicators including a first indicator indicative of the reference point or group of reference points of the subject for the first representation. The controller unit may be configured to determine the first indicator of the second representation based on the output of the verification function and/or the scanner data. The one or more indicators of the second representation may be based on the tracking data. The controller unit may be configured to include the one or more indicators or at least a part thereof in the second representation. The second representation may be different from the first representation.

The first representation and/or the second representation may comprise a model of the scanning subject. The first indicator indicative of the reference point or group of reference points of the subject for the first representation may be overlayed on the model of the scanning subject.

A plurality of different representations improves the operator's ability to monitor the scanning session and to change or adapt the scanning session in order to obtain an improved or optimum quality of the medical images obtained with the medical scanner.

The controller unit may be configured to update the first and/or the second representation with an update frequency of at least 1 Hz, such as less than 50 Hz, such as less than 20 Hz. The second representation may be provided in real-time or substantially in real-time, e.g. with a delay of less than 1 minute, less than 10 seconds, less than 1 second, less than 50 milliseconds, less than 10 milliseconds.

The second representation may comprise a notification signal, e.g. if the output is indicative of an erroneous scanning. The notification signal of the second representation may comprise one or more error indicators. The notification signal of the second representation may comprise blinking error indicator(s).

The scanning monitoring apparatus may comprise any sensor system capable of providing, or configured to provide, 3D surface representation data of an imaging region of the subject.

The scanning monitoring apparatus may comprise an image projector optionally configured to project one or a plurality of images onto an imaging region of the subject in the medical scanner. The tracking data may comprise image control data of the image projector. The image projector may be configured to project structured light onto an imaging region of the subject in the medical scanner.

The scanning monitoring apparatus may comprise a multiple or stereo-vision camera configured to obtain 3D surface representation data of an imaging region of the subject in the medical scanner. The tracking data may comprise the 3D surface representation data or at least a part thereof.

The scanning monitoring apparatus may comprise an image detector configured to detect images of a subject. The tracking data may comprise image data of the detected images.

The scanning monitoring apparatus operates before and/or during a scanning session of a (scanning) subject or patient.

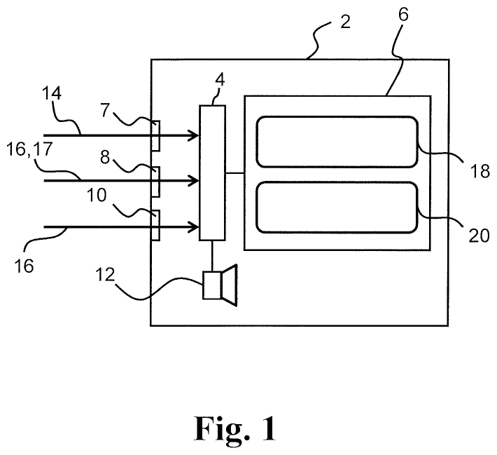

FIG. 1 illustrates an exemplary scanning monitoring apparatus for medical imaging, such as for biomedical imaging. The scanning monitoring apparatus 2 comprises a controller unit 4 and a display 6. Optionally, the scanning monitoring apparatus comprises a tracking interface 7 and a user interface 8, such as a keyboard and/or a pointing device (mouse or pen). The display may be a touch display configured as a user interface. The scanning monitoring apparatus may comprise a scanner interface 10 and a loudspeaker 12. The controller unit 4 is, during a scanning session, configured to obtain tracking data 14 of a subject in a medical scanner. The tracking data is indicative of subject motion of the subject. It is highly desired to compensate or correct for subject motion during a scanning session. The tracking data optionally comprises time series of surface point sets of a subject region of the subject. The surface point sets are optionally generated from depth sensors such as structured light systems, stereo vision systems, time-of-flight sensors (or extracted from image volumes of the medical scanner). Tracking data is acquired during medical scanning, such as magnetic resonance imaging (MRI), PET, CT, SPECT, MEG or combined scanning as PET/CT and MRI/PET.

The controller unit 4 is configured to obtain scanner data 16 indicative of operating parameters of the medical scanner, for example via the scanner interface 10 connected to the medical scanner and/or via the user interface 8 allowing the operator of the medical scanner to input necessary scanner data. The scanner data comprises a scanning type identifier indicative of the scanner type (PET, MRI, CET, or any combination). The controller unit 4 may be configured to obtain user input data 17 indicative of user input allowing the operator of the medical scanner to provide user input, e.g. comprising configuration parameters for the first representation and/or the second representation, such as color settings, point-of-view.

The scanner data 16 comprises one or more operating parameters of the medical scanner. Exemplary operating parameters of the medical scanner are:
Timing, e.g. scan acquisition on/off and/or start/stop
Scan session protocol, e.g. MRI/PET brain tumor examination
Scan parameters affecting the resulting spatial resolution and time resolution of the medical images. E.g. the individual MR image acquisitions may vary in motion sensitivity, for example as dependent on acquisition method: DWI, fMRI, structural MRI, etc. or scanner specifications including magnetic field strength bore size, etc. and acquisition method settings: spin echo, relaxation settings, k-space readout, etc.
Biomarker and contrast agent

The scanner data 16 comprises one or more scanning subject parameters of the subject that is scanned in the scanning session. Exemplary scanning subject parameters are:
Age
Gender
Weight
Examination (putative/suspected disease)
Diseases, e.g. tumor/lesion, Alzheimer's disease, neurotic diseases
Symptoms, e.g. pain, dementia, tremor
Consciousness level, e.g. sedated, awake, sleeping.

The controller unit 4 is configured to determine an output of a verification function based on the tracking data and the scanner data. For example, the output is set to a value indicative of erroneous scanning if a displacement of the scanning subject, a reference point of the scanning subject, or a reference point representing the scanning subject or a group of reference points of the scanning subject is larger than a threshold value and thus meets an error criterion. The controller unit 4 is configured to control the scanning monitoring apparatus according to the output of the verification function. For example, when the output is indicative of an erroneous scanning, the controller unit 4 is configured to determine and provide a notification signal in the form of an audio signal with the loudspeaker 12. Further, the controller unit is configured to determine a first representation 18 based on the tracking data 14, scanner data 16 and/or user input data 17. Further, the controller unit is configured to determine a second representation 20 based on the tracking data 14, scanner data 16 and/or user input data 17 and provide the representations 18, 20 to the display 6.

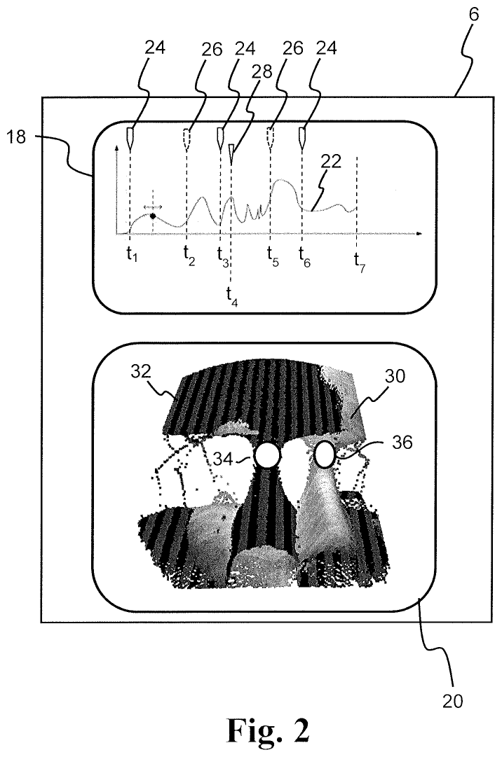

FIG. 2 shows a display with an exemplary first representation 18 and a second representation 20 provided by the controller unit 4 at time t7. The first representation 18 comprises a graphical representation 22 of displacement of a reference point of the subject as a function of time. The controller unit 4 has determined and included first indicators 24 indicative of start of scan acquisition at times t1, t3, and t6 based on scanner data 16. Further, the controller unit 4 has determined and included second indicators 26 indicative of end of scan acquisition at times t2 and t5 based on scanner data 16. The first indicators 24 are different from the second indicators 26. At time t4, the output of the verification function was indicative of an erroneous scanning (tracking data indicated a displacement larger than a threshold value while scanner data indicated that scan acquisition was on) and the controller unit determined and provided an error indicator 28 in the first representation. Further, the controller unit provided a first notification signal at time t4 in the form of an audio signal via loudspeaker 12, and a second notification signal at time t4 in the form of a blinking error indicator in the second representation 20. The notification signal of the second representation may comprise blinking error indicator(s). Despite the large displacement between t5 and t6 no notification signal or error indicator was provided, since the scanner data indicated that no scan acquisition took place, which also follows directly from the first indicators 24 and the second indicators 26.

The second representation 20 is based on the tracking data and the scanner data at time t7. The second representation 20 is different from the first representation 18 and comprises a surface representation 30 of a surface of the scanning subject. Further, the second representation may comprise a reference surface representation 32 of a surface of the scanning subject. Optionally, the second representation 20 comprises a first indicator 34 indicative of the reference point for the graphical representation 22 of the first representation 18. The controller unit determines the first indicator 34 of the second representation 20 based on the tracking data, the output of the verification function and/or the scanner data. Optionally, the second representation 20 comprises a second indicator 36 indicative of the position of the reference point for the graphical representation 22 compared to the surface representation 30. The controller unit determines the second indicator 36 of the second representation 20 based on the tracking data, the output of the verification function and/or the scanner data.

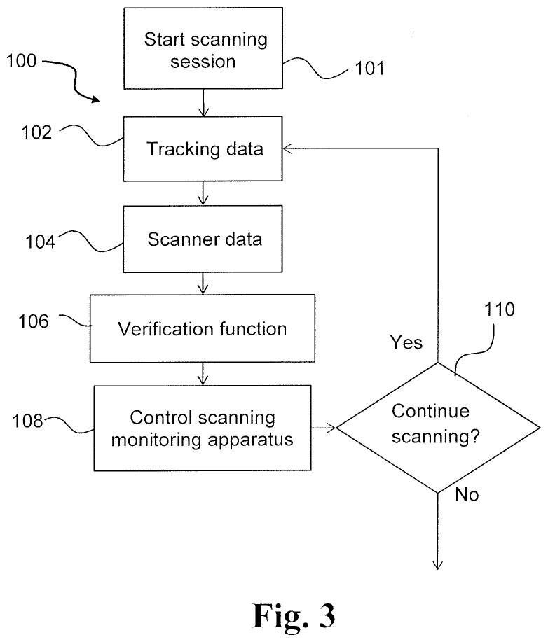

FIG. 3 is a flow-chart of an exemplary method of operating a scanning monitoring apparatus for biological/medical imaging, the scanning monitoring apparatus comprising a controller unit and a display. The method 100 comprises starting 101 a scanning session. After starting the scanning session, the method 100 comprises, during the scanning session, obtaining 102 tracking data of a subject in a medical scanner; obtaining 104 scanner data indicative of operating parameters of the medical scanner; determining 106 an output of a verification function based on the tracking data and the scanner data; and controlling 108 the scanning monitoring apparatus according to the output of the verification function. If the scanning is to continue 110, the method returns to obtaining 102 tracking data. Obtaining 102 tracking data and obtaining 104 scanner data may be performed in parallel and/or sequentially.

Obtaining 102 tracking data may comprise obtaining a 3D surface representation of a subject region generated from 3D/depth sensors such as structured light systems, stereo vision systems, time-of-flight sensors (or extracted from image volumes of the medical scanner). The subject region of interest in particular the face and/or the surface of the body part from which the medical scanner acquires data.

Tracking data is obtained during a scanning session, e.g. of medical imaging; magnetic resonance imaging (MRI), positron emission tomography (PET), CT, SPECT or combined scanners such as PET/CT and MRI/PET).

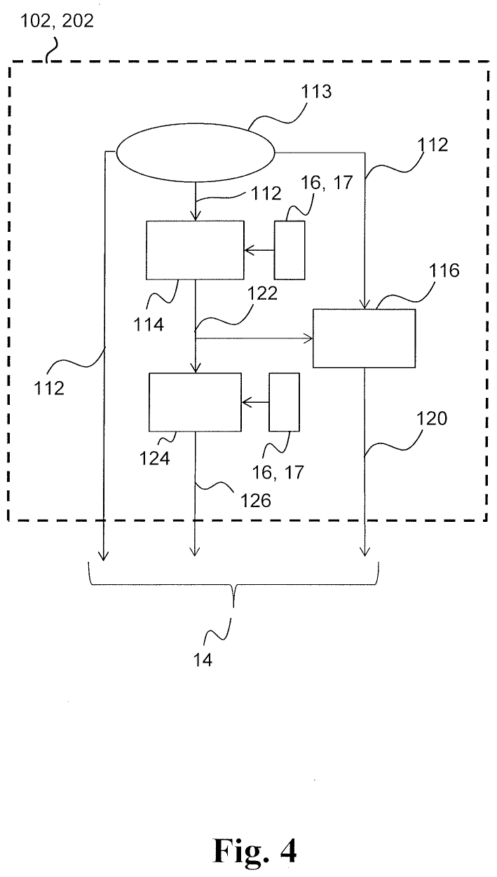

FIG. 4 schematically illustrates in more detail an example of obtaining tracking data. Raw tracking data 112 (3D representation, such as surface points) are obtained 113 and optionally weighted and/or filtered by weighting function and/or filtering function 114. Optionally, the quality of the raw tracking data (3D representation data, such as surface points) is verified in tracking data verification 116 outputting one or more quality parameters 120 indicative of tracking data quality as part of the tracking data. Filtered/weighted raw tracking data 122 may be input to tracking data verification 116. A displacement data function 124 determines displacement data 126 that are included in the tracking data. The displacement data function determines displacement data indicative of scanning subject displacement, e.g. in relation to one or more reference points or groups of reference points.

The displacement data function may determine displacement based on tracking data obtained at different points in times and related to predetermined reference points, thus, the displacement function may as input receive reference points, such as predetermined reference points, tracking data related to the reference points at a reference point in time and tracking data related to the reference points at an evaluation point in time. For example, the displacement may be given as the distance between given point(s) at time t1 and the same point(s) at time t2.

The input to the weighting/filtering function 114 may comprise one or more of 2D images, 3D point sets or 3D representation data (current point set, previous point set from which e.g. distances to neighbor points and point normals can be extracted), pre-defined region of interests (e.g. to mask out MR coils), model of the scan object. The weighting/filtering function 114 may be based on scanner data 16, e.g. one or more operating parameters of the medical scanner, such as a first operating parameter or a set of first operating parameters, wherein the first operating parameter(s) may be indicative of a scanning mode, a second operating parameter or a set of second operating parameters, wherein the second operating parameter(s) may be indicative of start of scan acquisition, and/or a third operating parameter or a set of third operating parameters, wherein the third operating parameter(s) may be indicative of end of scan acquisition. The weighting/filtering function 114 may be based on scanner data e.g. comprising one or more scanning subject parameters of the subject that is scanned in the scanning session.

The weighting/filtering function may comprise manual region of interest selection with binary weights. A semi-automated method may be applied to potentially only the reference based on distance neighbor clustering. Hereby, a verification step may be required to ensure that the data evaluated represents the object of interest.

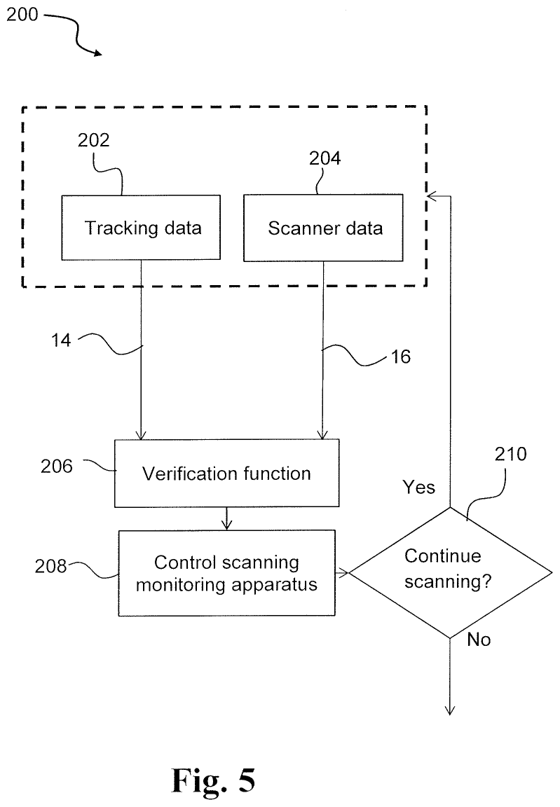

FIG. 5 illustrates operation 200 of an exemplary controller unit. The controller is configured to obtain 202 tracking data 14 including one or more of raw tracking data, displacement data, and one or more quality parameters. The controller unit is configured to obtain 202 scanner data 16. Further, the controller unit applies a verification function 206 and controls the scanning monitoring apparatus, e.g. by determining and optionally applying a notification scheme comprising one or more notification signals.

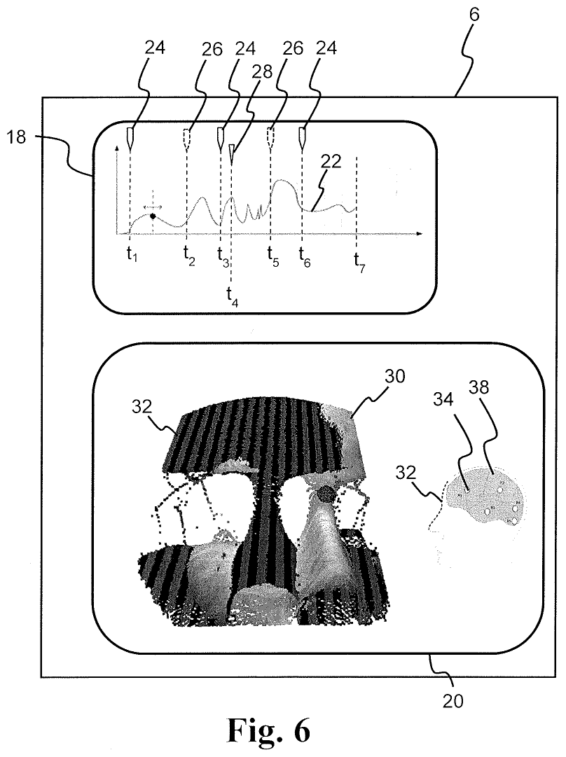

FIG. 6 schematically illustrates a display with exemplary first and second representations. The second representation 20 comprises a model representation 38 of the scanning subject. The first indicator 34 indicates the current position of the reference point for the first representation and is overlayed on the model representation 38. The model representation 38 may be included in the first representation 18 with an indicator indicating the current position of the reference point for the first representation overlayed on the model representation 38.

Also disclosed is scanning monitoring apparatus and method of operating a scanning monitoring apparatus according to the following items:

Item 1. A scanning monitoring apparatus for medical imaging, the scanning monitoring apparatus comprising a controller unit and a display, wherein the controller unit is, during a scanning session, configured to:
obtain tracking data of a subject in a medical scanner;
obtain scanner data indicative of operating parameters of the medical scanner;
determine an output of a verification function based on the tracking data and the scanner data; and
control the scanning monitoring apparatus according to the output of the verification function.

Item 2. Scanning monitoring apparatus according to item 1, wherein to control the scanning monitoring apparatus according to the output of the verification function comprises to provide a notification signal if the output is indicative of an erroneous scanning.

Item 3. Scanning monitoring apparatus according to item 2, wherein the scanning monitoring apparatus comprises a loudspeaker, and wherein to provide a notification signal comprises to provide an audio signal with the loudspeaker.

Item 4. Scanning monitoring apparatus according to item 3, wherein the controller unit is configured to select the audio signal from a plurality of audio signals based on the output of the verification function.

Item 5. Scanning monitoring apparatus according to any of items 2-4, wherein to provide a notification signal comprises to provide a visual alarm signal on the display.

Item 6. Scanning monitoring apparatus according to any of items 1-5, wherein to control the scanning monitoring apparatus according to the output of the verification function comprises to transmit a first control signal to the medical scanner, wherein the first control signal is indicative of stop scanning if the output is indicative of a first error.

Item 7. Scanning monitoring apparatus according to any of items 1-6, wherein to control the scanning monitoring apparatus according to the output of the verification function comprises to transmit a second control signal to the medical scanner, wherein the second control signal is indicative of scanning restart if the output is indicative of a second error.

Item 8. Scanning monitoring apparatus according to any of items 1-7, wherein to control the scanning monitoring apparatus according to the output of the verification function comprises to determine and provide a first representation based on the tracking data.

Item 9. Scanning monitoring apparatus according to any of items 1-8, wherein to control the scanning monitoring apparatus according to the output of the verification function comprises to determine and provide a second representation based on the tracking data.

Item 10. Scanning monitoring apparatus according to any of items 1-9, wherein the scanning monitoring apparatus comprises an image projector configured to project one or a plurality of images onto an imaging region of the subject in the medical scanner, and wherein the tracking data comprises image control data of the projector.

Item 11. Scanning monitoring apparatus according to any of items 1-9, wherein the scanning monitoring apparatus comprises any sensor system configured to provide 3D surface representation data of an imaging region of the subject in the medical scanner, and wherein the tracking data comprises the 3D surface representation data.

Item 12. Scanning monitoring apparatus according to any of items 1-10, wherein the scanning monitoring apparatus comprises an image detector configured to detect images of a subject, and wherein the tracking data comprises image data of the detected images.

Item 13. Scanning monitoring apparatus according to any of items 1-12, wherein the controller unit is further configured to obtain scanning subject parameters.

Item 14. Method of operating a scanning monitoring apparatus for medical imaging, the scanning monitoring apparatus comprising a controller unit and a display, wherein the method comprises, during a scanning session:
obtaining tracking data of a subject in a medical scanner;
obtaining scanner data indicative of operating parameters of the medical scanner;
determining an output of a verification function based on the tracking data and the scanner data; and
controlling the scanning monitoring apparatus according to the output of the verification function.

The use of the terms “first”, “second”, “third” and “fourth”, etc. does not imply any particular order, but are included to identify individual elements. Moreover, the use of the terms first, second, etc. does not denote any order or importance, but rather the terms first, second, etc. are used to distinguish one element from another. Note that the words first and second are used here and elsewhere for labelling purposes only and are not intended to denote any specific spatial or temporal ordering. Furthermore, the labelling of a first element does not imply the presence of a second element and vice versa.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications and equivalents.

### LIST OF REFERENCES

2 scanning monitoring apparatus
4 controller unit
6 display
7 tracking interface
8 user interface
10 medical scanner interface
12 loudspeaker
14 tracking data
16 scanner data
17 user input data
18 first representation
20 second representation
22 graphical representation
24 first indicator of first representation
26 second indicator of first representation
28 error indicator
30 surface representation
32 reference surface representation
34 first indicator of the second representation
36 second indicator of the second representation
38 model representation of scanning subject
100 method of operating a scanning monitoring apparatus for medical imaging
101 start scanning session
102 obtaining tracking data
104 obtaining scanner data
106 determining an output of a verification function
108 controlling the scanning monitoring apparatus
110 continue scanning?
112 raw tracking data
113 obtain raw tracking data
114 apply weighting and/or filtering
116 tracking data verification
120 quality parameter(s) indicative of tracking data quality
122 filter/weighting function filtered/weighted raw tracking data
124 displacement data function
126 displacement data
200 operation of controller unit
202 obtain tracking data
204 obtain scanner data
206 apply verification function
208 control scanning and tracking monitoring apparatus
210 decide if scanning session is stopped

1. A scanning monitoring apparatus for medical imaging, the scanning monitoring apparatus comprising a controller unit and a display, wherein the controller unit is, during a scanning session, configured to:
obtain tracking data of a subject in a medical scanner, wherein to obtain tracking data includes to determine one or more quality parameters indicative of tracking data quality and to include the one or more quality parameters in the tracking data;
obtain scanner data indicative of operating parameters of the medical scanner;
determine an output of a verification function based on the tracking data and the scanner data; and
control the scanning monitoring apparatus according to the output of the verification function.

2. Scanning monitoring apparatus according to claim 1, wherein the output of the verification function is indicative of corrupted tracking data if a quality parameter of the one or more quality parameters indicative of tracking data quality does not fulfil a tracking data quality criterion.

3. Scanning monitoring apparatus according to claim 1, wherein the output of the verification function is indicative of high quality of tracking data if a quality parameter of the one or more quality parameters indicative of tracking data quality fulfils a tracking data quality criterion.

4. Scanning monitoring apparatus according to claim 1, wherein to control the scanning monitoring apparatus according to the output of the verification function includes to determine and provide a first representation based on the tracking data.

5. Scanning monitoring apparatus according to claim 4, wherein the first representation includes a graphical representation of displacement of the subject as a function of time, and
wherein the graphical representation of displacement includes a first graphical representation of total 3D displacement in distance units, a second graphical representation of first displacement along a first axis as a function of time, a third graphical representation of second displacement along a second axis as a function of time, and a fourth graphical representation of third displacement along a third axis as a function of time.

6. (canceled)

7. Scanning monitoring apparatus according to claim 4, wherein the controller unit is configured to determine one or more indicators based on the output of the verification function, the scanner data, and the tracking data, and wherein the controller unit is configured to include the one or more indicators or at least a part thereof in the first representation, and wherein the one or more indicators comprises an error indicator or a set of error indicators indicative of erroneous scanning.

8. (canceled)

9. Scanning monitoring apparatus according claim 7, wherein the one or more indicators indicate at which time the corresponding event occurred.

10. Scanning monitoring apparatus according to claim 4, wherein to control the scanning monitoring apparatus according to the output of the verification function includes to determine and provide a second representation based on the tracking data, wherein the second representation is different from the first representation, and
wherein the second representation includes a surface representation of a surface of the scanning subject and a reference surface representation of a surface of the scanning subject and/or
wherein the second representation includes one or more indicators including a first indicator indicative of a reference point or group of reference points of the subject for the first representation.

11. (canceled)

12. (canceled)

13. Scanning monitoring apparatus according to claim 10, wherein the controller unit is configured to determine the first indicator of the second representation based on the output of the verification function, the scanner data, and the tracking data.

14. Scanning monitoring apparatus according to claim 1, further comprising an image projector configured to project one or a plurality of images onto an imaging region of the subject in the medical scanner, and wherein the tracking data includes image control data of the image projector.

15. Scanning monitoring apparatus according to claim 1, wherein to control the scanning monitoring apparatus according to the output of the verification function includes to provide a notification signal if the output is indicative of an erroneous scanning.

16. Scanning monitoring apparatus according to claim 15, further comprising a loudspeaker, and wherein to provide a notification signal includes to provide an audio signal with the loudspeaker.

17. Scanning monitoring apparatus according to claim 16, wherein the controller unit is configured to select the audio signal from a plurality of audio signals based on the output of the verification function.

18. Scanning monitoring apparatus according to claim 15, wherein to provide a notification signal includes to provide a visual alarm signal on the display.

19. Scanning monitoring apparatus according to claim 1, wherein to control the scanning monitoring apparatus according to the output of the verification function includes to transmit a first control signal to the medical scanner, wherein the first control signal is indicative of stop scanning if the output is indicative of a first error.

20. Scanning monitoring apparatus according claim 1, wherein to control the scanning monitoring apparatus according to the output of the verification function includes to transmit a second control signal to the medical scanner, wherein the second control signal is indicative of scanning restart if the output is indicative of a second error.

21. Scanning monitoring apparatus according to claim 1, further comprising any sensor system configured to provide 3D surface representation data of an imaging region of the subject in the medical scanner, and wherein the tracking data includes the 3D surface representation data.

22. Scanning monitoring apparatus according to claim 1, further comprising an image detector configured to detect images of a subject, and wherein the tracking data includes image data of the detected images.

23. Scanning monitoring apparatus according to claim 1, wherein the controller unit is further configured to obtain scanning subject parameters.

24. Method of operating a scanning monitoring apparatus for medical imaging, the scanning monitoring apparatus including a controller unit and a display, wherein the method comprises, during a scanning session:
obtaining tracking data of a subject in a medical scanner, wherein obtaining tracking data includes determining one or more quality parameters indicative of tracking data quality and including the one or more quality parameters in the tracking data;
obtaining scanner data indicative of operating parameters of the medical scanner;
determining an output of a verification function based on the tracking data and the scanner data; and
controlling the scanning monitoring apparatus according to the output of the verification function.
